(19) United States
(12) Patent Application Publication
(10) Pub. No.: US 2007/0135686 A1
(43) Pub. Date: Jun. 14, 2007

(54) TOOLS AND METHODS FOR EPICARDIAL ACCESS

(76) Inventors: John Crayton Pruitt JR., Largo, FL (US); Robert R. Lazzara, Tampa, FL (US); Kenneth Wayne Grace, Oak Ridge, TN (US); Adam L. Berman, Edina, MN (US)

Correspondence Address: MERCHANT & GOULD PC, P.O. BOX 2903, MINNEAPOLIS, MN, 55402-0903, US

(21) Appl. No.: 11/609,431

(22) Filed: Dec. 12, 2006

Related U.S. Application Data
(60) Provisional application No. 60/750,223, filed on Dec. 14, 2005.

(51) Int. Cl. A61B 1/32

(52) U.S. Cl. 600/214

(57) ABSTRACT

A retractor tool has a hollow shaft with a proximal end and a distal end. The shaft has an internal bore sized to slidable receive an elongated tool of predetermined dimensions with a distal end of said tool exposed through a distal end of the bore. Such tool may be a visualization tool or other tool used in surgery. A first pivoting jaw and a second jaw are both secured to the distal end of the shaft to pivot about first and second pivot axes perpendicular to an axis of the shaft. The jaws pivot between open and closed positions. In the closed positions, opposing surfaces of the jaws define a distal end of the bore extending completely between the jaws.

### CROSS-REFERENCE TO RELATED APPLICATION

This patent application claims priority to U.S. Provisional Patent Application Ser. No. 60/750,223 filed Dec. 14, 2005.

### BACKGROUND OF THE INVENTION

1. Field of the Invention

This invention pertains to tools and methods for epicardial access. More particularly, this invention pertains to tools and methods for forming a dissection through a pericardial reflection at a transverse sinus of a patient's heart during an endoscopic procedure.

2. Description of the Prior Art

A number of different methods have been suggested for creating lesions around pulmonary veins for the purpose of treating atrial fibrillation. Examples of these can be found in U.S. Pat. Nos. 6,474,340; 6,805,129; 6,161,543; 6,314,962; 6,314,963; 6,474,240 and 6,949,095. Also, such procedures and related tools are described in U.S. Patent Application Publication Nos. US 2003/0029462; US 2004/0260278; US 2004/0054263; US 2004/147912; US 2004/0102771 and US 2002/0087151.

The foregoing patent applications and patents teach placing an ablation element on an epicardial surface of the heart. Preferably, a complete lesion is formed surrounding pulmonary veins to isolate the pulmonary veins in a so-called MAZE procedure. Ablation elements come in a wide variety of forms and can include ultrasound, radio frequency ablation, laser ablation as well as diffused light ablation.

While MAZE procedures can be performed in so-called open chest procedures, atrial fibrillation treatments are most preferably performed through minimally invasive or endoscopic procedures in the thoracic cavity (“thoracoscopic” procedures). In such procedures, several ports are formed through the patient's chest in small incisions between the ribs to provide access for visualization instruments and surgical tools.

Minimally invasive cardiac procedures present challenging surgical obstacles requiring novel techniques to safely accomplish a procedure. For example, it is desirable to provide access to the epicardial surface surrounding the pulmonary veins on an approach directed from the right side of the patient's heart. Mainly, a right-side approach permits visualization of important anatomical landmarks as well as having anatomical features which help guide ablation instruments around the pulmonary veins.

When approaching the epicardial surface of the heart surrounding the pulmonary veins from the right side, a surgeon encounters a pericardial reflection at the transverse sinus. The pericardial reflection is tissue of the pericardium extending up from the epicardial surface in a region where the superior vena cava or inferior vena cava pass over the epicardial surface in close proximity to a pulmonary artery. A dissection must be made through the pericardial reflection in order to obtain access to the pericardial space in the vicinity of the pulmonary veins as well as provide access for visualizing important anatomical landmarks (such as the left atrial appendage) when performing the procedure.

Forming an incision through the pericardial reflection beneath the superior vena cava is a delicate procedure. Extremely important anatomical features (such as pulmonary arteries or aorta) reside behind the pericardial reflection obscured from vision of the surgeon. Excessive advancement of a dissection tool through the pericardial reflection presents risk of injury to such structures. It is an objection of the present invention to provide a method and tool for assisting in a surgical procedure and more particularly for assisting in forming an incision through the pericardial reflection at the transverse sinus.

### SUMMARY OF THE INVENTION

According to a preferred embodiment of the present invention, a method and apparatus are disclosed for forming an incision in a tissue between anatomical features of a patient. The apparatus includes a retractor tool having a hollow shaft with a proximal end and a distal end. The shaft has an internal bore sized to slidable receive an elongated tool of predetermined dimensions with a distal end of said tool exposed through a distal end of the bore. Such tool may be a visualization tool or other tool used in surgery. A first pivoting jaw and a second jaw are both secured to the distal end of the shaft to pivot about first and second pivot axes perpendicular to an axis of the shaft. The jaws pivot between open and closed positions. In the closed positions, opposing surfaces of the jaws define a distal end of the bore extending completely between the jaws.

The method includes inserting the retractor tool into a patient with the jaws in the closed position. A visualization tool is placed within the bore to visualize an advancement of a distal tip of the retractor tool. The retractor tool is advanced toward the tissue with the jaws positioned between the anatomical features. The jaws are opened with the jaws urging a separation between the anatomical features and applying a tension to the tissue.

### BRIEF DESCRIPTION OF THE DRAWINGS

FIG. 1 is a side elevation view of a retractor of the present invention with jaws moved toward a pericardial reflection;

FIG. 1A is the view of FIG. 1 with jaws urged against the pericardial reflection to initiate application of tension to the pericardial reflection;

FIG. 1B is the view of FIG. 1A with retractor jaws moved to an open position;

FIG. 2 is a perspective view of a combination of the present invention including a retractor according to the present invention and a prior art endoscope;

FIG. 3 is a side elevation view of a distal end of the retractor of FIG. 2 with retractor's jaws shown in the closed position;

FIG. 4 is a perspective view of the distal tip of FIG. 3;

FIG. 5 is the view of FIG. 4 with the jaws shown in the open position;

FIG. 6 is a perspective view of a distal end of an alternative embodiment of a retractor element with a 4-jaw configuration;

FIG. 7 is an end view of a distal end of the retractor element of FIG. 6;

FIG. 8 is a view taken along lines 8-8 of FIG. 7;

FIG. 9 is a perspective view similar to FIG. 6 showing a still further alternative embodiment with a 5-jaw configuration in a closed position;

FIG. 10 is the view of FIG. 9 with jaws shown in an open position;

FIG. 11 is a perspective view of a further embodiment of the apparatus of FIG. 1 with an actuating handle;

FIG. 12 is a side elevation view of the retractor of FIG. 11;

FIG. 13 is a top plan view of the retractor of FIG. 11;

FIG. 14 is a proximal end view of the retractor of FIG. 11;

FIG. 15 is a view taken along line 15-15 of FIG. 13;

FIG. 16 is a view taken along line 16-16 of FIG. 12; and

FIG. 17 is a view taken along line 17-17 of FIG. 12.

### DESCRIPTION OF THE PREFERRED EMBODIMENT

With reference now to the various drawing figures in which identical elements are numbered identically throughout, a description of a preferred embodiment of the present invention will now be provided.

While human anatomy may vary from patient to patient, the superior vena cava overlies and is spaced from the left atrium. A pulmonary artery passes beneath the superior vena cava and over the left atrium. The space defined between the superior vena cava, left atrium, and pulmonary artery is blocked by a pericardial reflection. The pericardial reflection is pericardial tissue extending from the left atrium to the underside of the superior vena cava. To obtain access to the epicardial surface of the heart surrounding the pulmonary veins, an incision must be made through the pericardial reflection.

In a thoracoscopic procedure, such an incision can made by forming two ports between the ribs of the patient on the right side. The first port provides for visualization tools such as an endoscope. The second port permits advancement of a dissection tool to the pericardial reflection.

In its natural state, the pericardial reflection is a pliant fibrous tissue. Forming the dissection through the pericardial reflection may require piercing the pericardial reflection or moving the dissection tip of a dissection tool laterally against the surface of the pericardial reflection.

Unfortunately, due to the nature of the compliant tissue of the pericardial reflection, the dissection tool may urge the pericardial reflection and, hence, the tip of the deflection tool toward the left side of the heart in close proximity to anatomical features such as pulmonary arteries or the aorta. If the dissection tool were to advance through the pericardial reflection and perforate any of these vessels, serious consequences can arise greatly complicating the surgical procedure. However, once an incision is safely formed through the pericardial reflection, the surgeon has direct visualization of these features as well as other anatomical landmarks important for the continuation of the procedure.

Applicants have found that the incision through the pericardial reflection can be more safely performed if tension is applied to the pericardial reflection during the dissection procedure. The apparatus and method of the present invention are directed to a tool for lifting the superior vena cava relative to the left atrium and thereby providing tension on the tissue of the pericardial reflection prior to forming an incision through the pericardial reflection with a dissection tool.

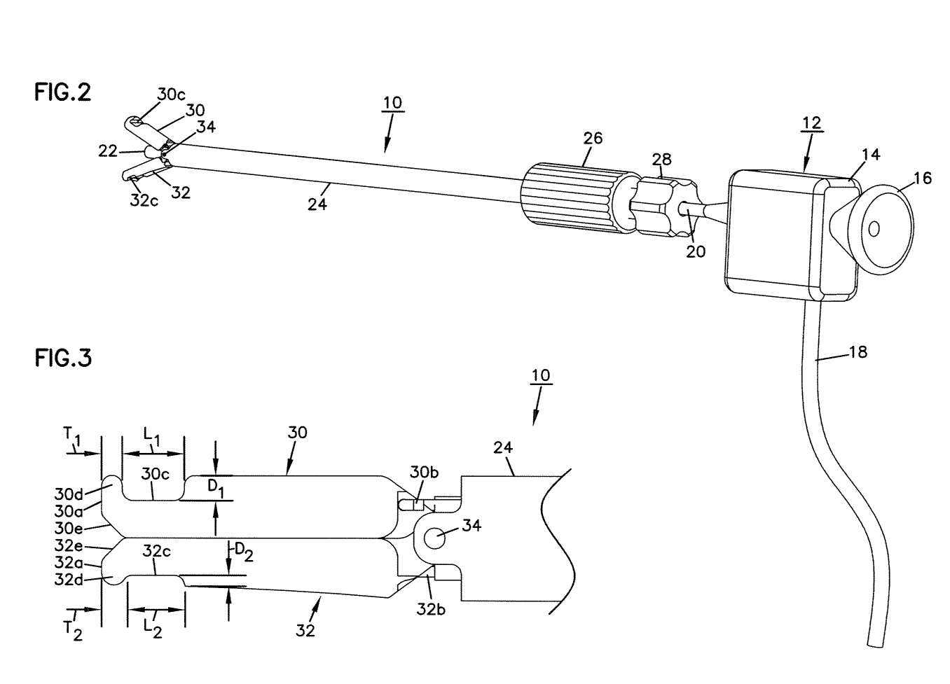

The apparatus of the present invention includes a novel combination of a novel retractor 10 in combination with a prior art endoscope 12. The endoscope 12 includes a proximal end 14 having a view port 16 and an optical fiber input 18. The endoscope 12 also has a distal fiber 20 of predetermined length and diameter. In a preferred embodiment, an endoscope 12 having a distal fiber 20 with a diameter of 5 millimeters and a length of 45 centimeters is preferred. The distal fiber 20 terminates at a fiber tip 22 (shown in FIGS. 2 and 5).

The retractor 10 includes a hollow tubular body 24 and a proximal handle 26 with an adjustment knob 28. Preferably, the tubular body 24 has an outer diameter of 10 millimeters so that it may be passed through conventional endoscopic cannula and directed toward the heart in a thoracoscopic procedure. Also, the body 24 has an inner diameter greater than the diameter of the endoscope fiber 20.

The tubular body 24 is preferably rigid. It may be made of plastic or any other biocompatible material suitable for medical use such as stainless steel. At its distal end, the tubular body 24 carries upper and lower retracting jaws 30, 32.

Shown best in FIG. 3, the retracting jaws 30, 32 are each generally configured as semi-cylindrical elements. Accordingly, when joined in a collapsed state (shown in FIG. 3), the jaws 30, 32 combine to form a generally cylindrical configuration. Preferably, this cylinder has a maximum outside diameter of 10 millimeters to permit advancement of the collapsed jaws 30, 32 through the same cannula as the tubular member 24.

Each of the jaws 30, 32 terminates at a flat axial face 30a, 32a which is in a plane substantially perpendicular to the cylindrical axis of the collapsed jaws 30, 32. Proximal ends of the jaws 30, 32 are hinged to the tubular body 24 at a hinge point 34. Each of the jaws 30, 32 are connected to the adjustment knob 28 by push rods 30b, 32b.

The upper jaw 30 includes a transverse detent 30c. In a preferred embodiment, the detent 30c has a depth D₁ of 2 millimeters, and axial length L₁ of 4.5 millimeters. Such dimensions are selected for at least a portion of the superior vena cava SVC of an adult human to reside within the detent 30c. The lower jaw 32 has a transverse detent 32c with a depth D₂ and a axial length L₂ of 1 millimeters and 4.5 millimeters, respectively.

The detents 30c, 32c are spaced from the axial faces 30a, 32a by a wall thickness T₁, T₂, each equal to 1.5 millimeters. The separating walls 30d, 32d are rounded to present smooth atraumatic surfaces for advancing the jaws towards the heart. Sidewalls of the jaws 30, 32 are chamfered as at 30e, 32e illustrated in FIG. 3.

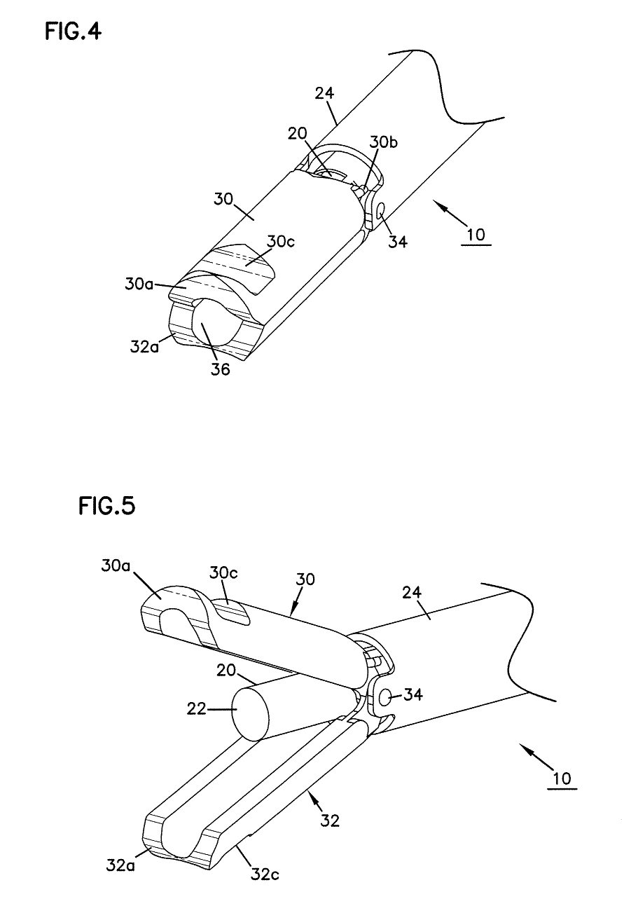

Interior surfaces of the jaws 30, 32 define a circular diameter bore 36 (FIG. 4) having a diameter at least as great as the diameter of a distal fiber 20 of endoscope 12. Since the length of the endoscope fiber 20 is greater than the length of the retractor 10, the fiber 20 may be advanced through the bore of the tubular body 20 and the bore 36 for fiber tip 22 to protrude beyond the axial faces 30a, 32a.

The push rods 30b, 32b are connected by linkages known in the art to the adjustment member 28. Rotation of the adjustment knob 28 permits opening or closing the jaws 30, 32 in a closed positioned shown in FIGS. 3 and 4 to an open position shown in FIG. 5.

With the construction thus described, the endoscope fiber 20 may be placed within the retractor 10 with the distal tip 22 at or protruding beyond the axial faces 30a, 32a. A combination of the endoscope 12 and retractor 10 may then be advanced through a cannula (not shown) positioned through an intercostal space of the patient and advanced towards the heart.

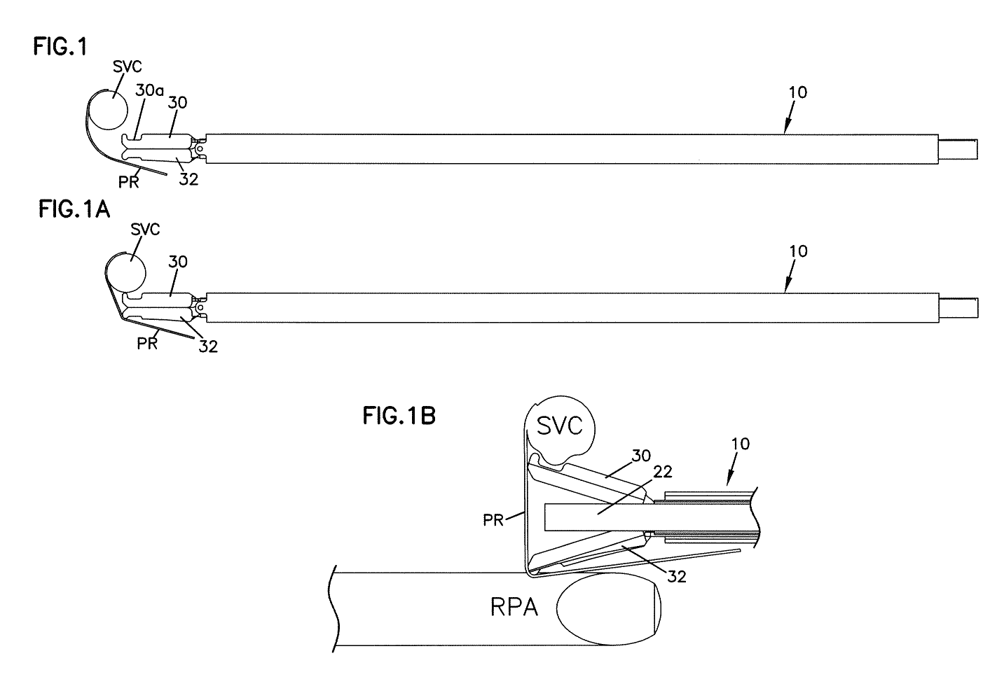

In a preferred embodiment, the apparatus is directed towards the right side of the heart with advancing of the axial faces 30a, 32b against the pericardial reflection beneath the superior vena cava illustrated in FIGS. 1 and 1A. The size of the pericardial reflection PR is such that the axial faces 30a, 32a (having a combined diameter of 10 mm when the jaws 30, 32 are closed) may be urged against the pericardial reflection PR with the upper detent 30a opposing the superior vena cava SVC.

Upon further urging of the axial faces 30a, 32a against the pericardial reflection PR (FIG. 1A), the superior vena cava SVC may roll over the upper rounded wall 30d and be received into the detent 30c. Throughout this process, accurate positioning of the axial faces 30a, 32a may be directly observed by reason of the endoscope distal tip 22 having a clear field of view through the bore 36.

Upon desired positioning of the faces 30a, 32a against the pericardial reflection PR, the adjustment knob 28 may be rotated to urge the jaws 30, 32 to the open position of FIG. 5. Such urging lifts the superior vena cava SVC relative to the left atrium LA and places the tissue of the pericardial reflection PR under tension (as shown in FIG. 1B which also shows a right pulmonary artery RPA in schematic form relative to the pericardial reflection PR and superior vena cava SVC).

To enhance the function of the present invention, the surfaces 30a, 32a and 30d, 32d may be made of material selected to enhance friction relative to the pericardial tissue. For example, they may be coated with a fabric or other material having enhanced friction against the pericardial reflection PR relative to smooth plastic or stainless steel.

When the pericardial reflection is under tension by the open jaws 30, 32, the jaws have opposing surfaces which present an open area into the interior of the bore 36. Accordingly, a dissection tool 100 may be advanced through a separate cannula (not shown) with the dissection tip 102 introduced between the jaws 30, 32 by passing the tip of the dissection tool between the sides of the open jaws. The dissection tip 102 may then be used to form a dissection in the pericardial reflection PR. Since the pericardial reflection PR is under tension, the resection is more easily performed with reduced risk of advancement of the dissection tip beyond the pericardial reflection PR and against anatomical structures on the opposite side of the pericardial reflection PR. Throughout this process, the dissection can be observed directly through the endoscope 12 which has its distal tip 22 directly opposing the tensioned pericardial reflection PR for direct observation.

After performing such a dissection, the adjustment member 28 may be rotated to close the jaws 30, 32 such that the retractor 10 and endoscope 12 may be removed from the patient. Alternatively, when in the closed position, the axial faces 30a, 32a may be advanced through the incision formed in the pericardial reflection PR and the jaws reopened to permit visualization of anatomical structures beyond the pericardial reflection.

As an alternative to providing a dissection tool through a separate cannula, the endoscope 12 may be removed from the retractor 10. A dissection tool then may be advanced through the tubular body 24 with its dissection tip forming the dissection through the pericardial reflection.

With apparatus and method thus described, a dissection of the pericardial reflection can be performed with greater safety and reliability.

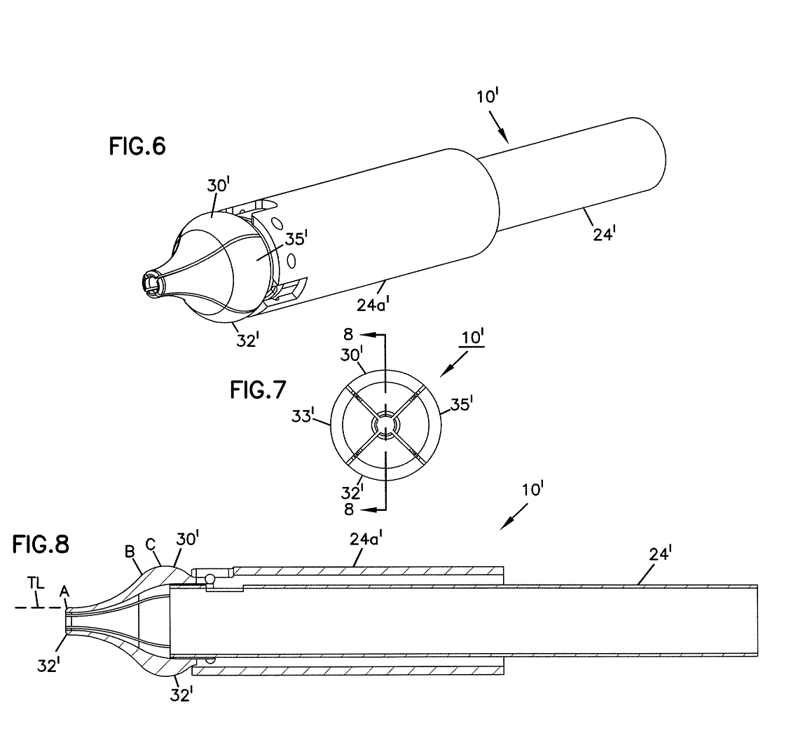

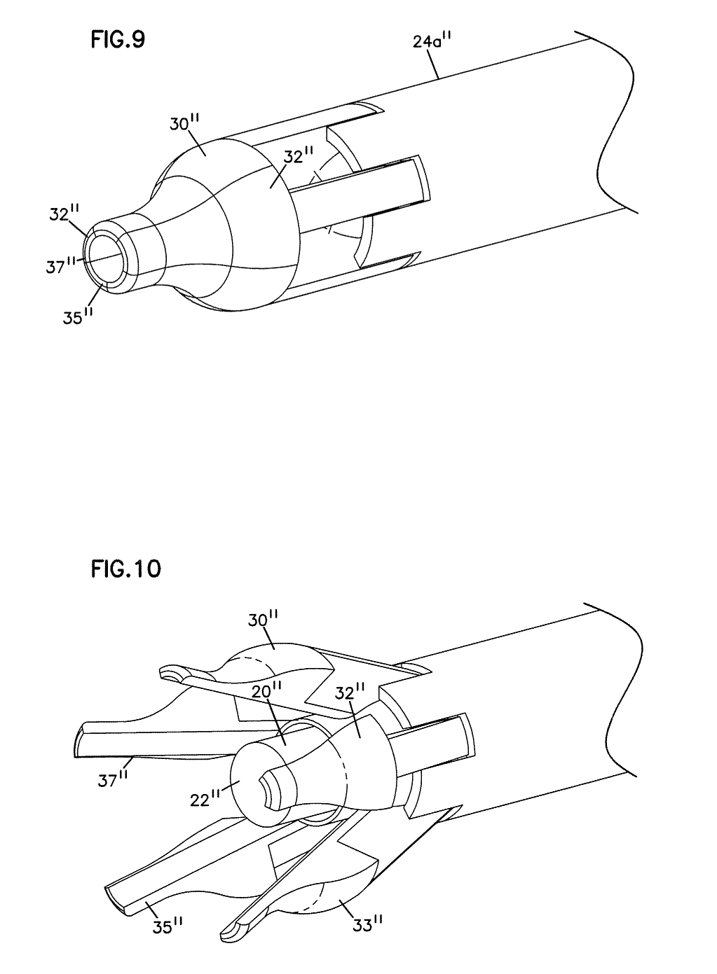

The retractor 10 in the forgoing embodiments has two opposing jaws 30, 32 which move in opposite directions. As a result, the tensioned area of the pericardial reflection PR is generally oval in configuration. FIGS. 6-10 illustrate an alternative embodiment to form a more circular configuration. Further, the embodiment of these figures also act to move the pulmonary artery away from the dissection area. Elements in common with the previous embodiment are numbered identically except for the addition of an apostrophe (in FIGS. 6-8) or double apostrophe (FIGS. 9 and 10) to distinguish the embodiments.

The retractor 10′ of FIGS. 6-8 includes a rigid inner tubular body 24′ surrounded by a sliding sleeve 24a′. Jaws 30′, 32′, 33′ and 35′ are arranged at the distal tip of the tube 24′. The embodiment of FIGS. 9-10 is identical except five jaws 30″, 32″, 33″, 35″, 37″ are shown.

In a collapsed state shown in FIGS. 6-9, the combined jaws have an ogee configuration (i.e., S-shaped in profile) with a narrow tip 32′, 32″ having an outer surface with a tangent line TL at end A generally parallel to the axis of the tube 24′ and advancing in increasing diameter to an inflection point B at which point the rate of increase of the diameter decreases to a maximum diameter at point C.

Spring mechanisms bias the jaws to the open position (FIG. 10). The sleeve 24a′, 24a″ when advanced to a fully deployed position retains the jaws against the bias in the closed position. Retraction of the sleeve 24a′, 24a″ permits the jaws to open in response to their bias. Between tip A and inflection point B, tissue can slide from point A to point B but resists sliding beyond point B.

The opposing jaws are hollow such that a fiber from an endoscope may be advanced through the hollow inner tubular body 24′, 24″ and permit inspection through the distal tip of the opposing jaws in both open and closed positions.

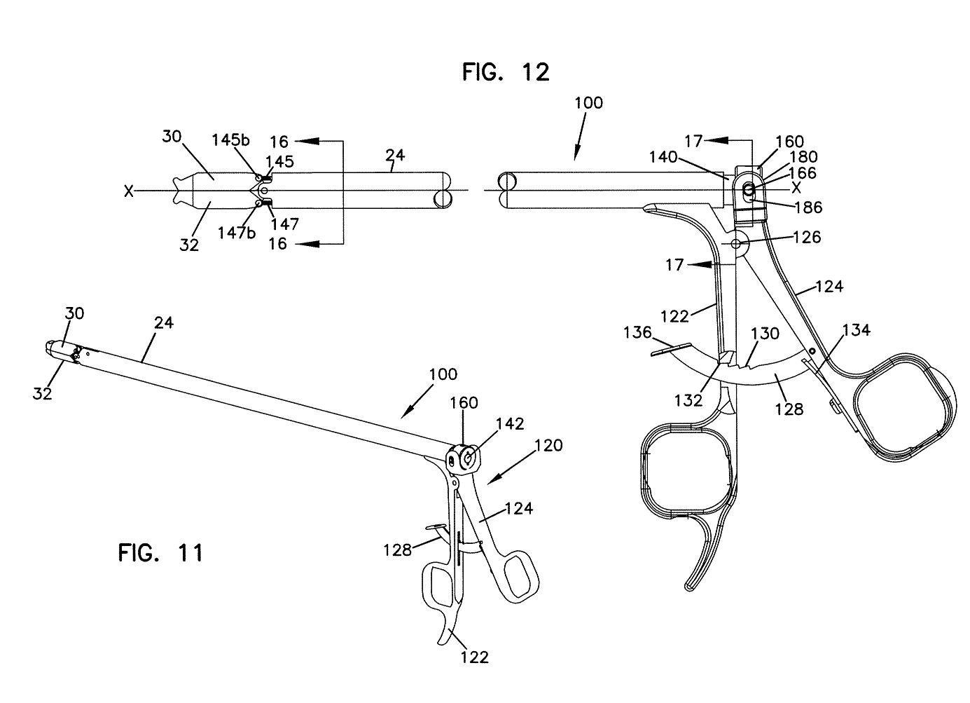

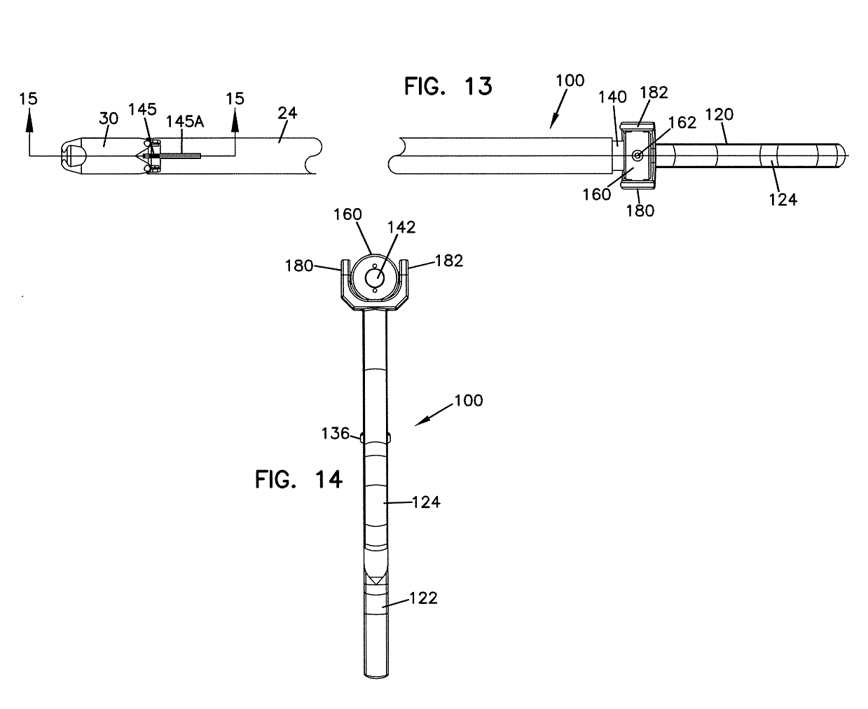

FIGS. 11-17 illustrate a most preferred embodiment of the present invention. Elements in common with those of FIGS. 1A and 3 are similarly numbered.

In FIGS. 11-17, the retractor 100 includes a tubular body 24 terminating at jaws 30, 32, as previously described. Preferably, the tube 24 is a straight cylinder. When the jaws 30, 32 are in the closed position (FIG. 12) the jaws 30, 32 are contained within a cylindrical extension of the tube 24. As such, the jaws 30, 32 pass through an incision (or cannula) sized to receive the tube 24.

In the figures, the retractor 100 includes a handle 120 at a proximal end of the tube 24. The handle 120 includes a stationary handle 122 ridgedly fixed to the tube 24. A movable handle 124 is pivotally connected to the stationary handle 122 at pivot point 126. Relative positioning of the movable handle 124 and stationary handle 122 may be fixed by a ratchet arm 128 having notches 130 which engage a surface 132 on the stationary handle 122. Element 128 is urged into locked engagement by a spring 134. A surgeon may urge the element 128 out of engagement by pressing on a tab 136 to permit relative movement between the handles 124, 122.

The tube 24 contains an inner tube 140. Inner tube 140 defines a bore 142 aligned with the bore 36. The bore 142 exposed through the proximal end of the retractor 100 as shown in FIG. 14. As previously described, the bore 142 is sized to be equal in size to bore 36 to permit sliding passage of a tool of predetermined dimensions through the bores 142, 36. Such tools may include a visualization tool such as an endoscope or a surgical tool such as dissection tool or other tools commonly used in thoracoscopic or laparoscopic procedures. As used herein, the term “tool” is intended to include any device which can be passed through bores 142, 36 as part of a surgical procedure. For example, ablation tools such as guided cardiac ablation tools described in US patent application publication numbers US 2006/0084960 published Apr. 20, 2006 and US 2005/0182392 A1 published Aug. 18, 2005 (both incorporated herein by reference).

The inner tube 140 has an outer diameter which abuts an inner diameter of outer tube 24. The inner tube 140 is slidable relative to the outer tube 24 along a longitudinal axis X-X common to both of tubes 24, 140.

On diametrically opposite upper and lower ends of the inner tube 140, the inner tube 140 has bores 144, 146 in the sidewall of tube 140 running the length of tube 140. The bores 144, 146 are sized to receive rods 145, 147 which extend the length of the tube 140.

The distal ends of the rods 145, 147 are connected to the upper and lower jaws 30, 32 at pivot points 145b, 147b. At the distal end, the tube 24 has slots 145a, 147a to accommodate movement of the rods 145, 147 as the jaws 30, 32 pivot.

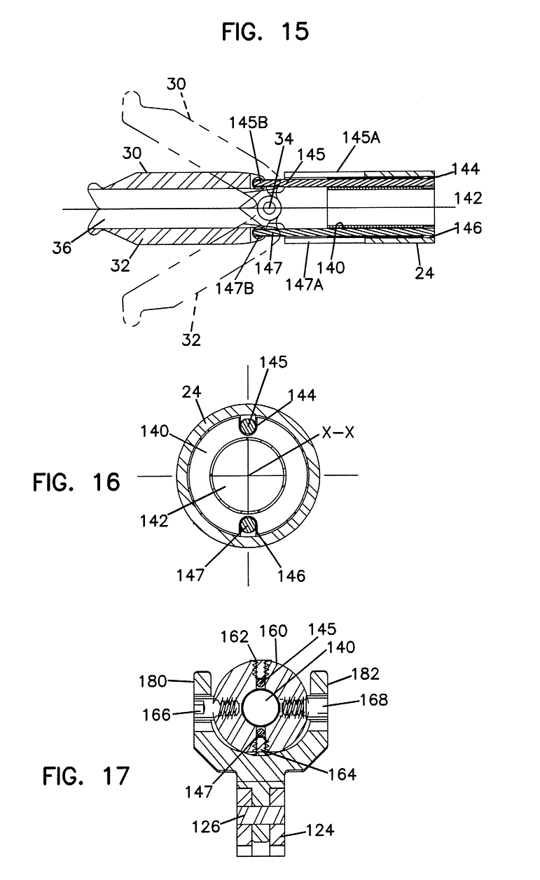

A proximal end of the inner tube 140 and rods 144, 147 are received within a hub 160. They are secured in place by set screws 162, 164. Lateral sides of the hub 160 include pins 166, 168 received within elongated slots formed in sidewalls 180, 182 of the handle 124. Only slot 186 in sidewall 180 is shown in FIG. 12. It will be appreciated that the opposite sidewall 182 is identically formed. Accordingly, when the handle 124 is moved towards handle 122, the hub 160 is moved proximally drawing the tube 140 in rod 145, 147 proximally resulting in opening of the jaws 30, 32 as illustrated by the phantom lines in FIG. 15.

By the forgoing, it has been shown how the objects of the invention have been attained in the preferred embodiment. Modifications and equivalents of the disclosed concepts such as those as readily occur to one of ordinary skill in the art are intended to be included within the scope of the claims which are appended hereto.

What is claimed is:

1. A retraction tool comprising:
a hollow shaft having a proximal end and a distal end and having an internal bore sized to slidable receive an elongated tool of predetermined dimensions with a distal end of said tool exposed through a distal end of said bore;
a first pivoting jaw and a second jaw both secured to said distal end of said shaft to pivot about first and second pivot axes perpendicular to an axis of said shaft;
said jaws pivotable between open and closed positions, in said closed positions, opposing surfaces of said jaws defining a distal end of said bore extending completely between said jaws for said bore to communicate with a exterior at a distal end of said jaws.

2. A tool according to claim 1 wherein, in the closed position, an external geometry of the jaws is contained with an extended external geometry of the shaft.

3. A tool according to claim 1 wherein, in the open position, the jaws do not obstruct the bore.

4. A tool according to claim 1 wherein the jaws present atraumatic distal tips.

5. A method for forming an incision in a tissue between anatomical features comprising:
selecting a retractor tool having:
a hollow shaft having a proximal end and a distal end and having an internal bore sized to slidable receive an elongated tool of predetermined dimensions with a distal end of said tool exposed through a distal end of said bore;
a first pivoting jaw and a second jaw both secured to said distal end of said shaft to pivot about first and second pivot axes perpendicular to an axis of said shaft;
said jaws pivotable between open and closed positions, in said closed positions, opposing surfaces of said jaws defining a distal end of said bore extending completely between said jaws for said bore to communicate with a exterior at a distal end of said jaws;
inserting said retractor tool into a patient with the jaws in the closed position;
placing a visualization tool within said bore to visualize an advancement of a distal tip of the retractor tool;
advancing the retractor tool toward the tissue with said jaws positioned between the anatomical features;
opening the jaws with the jaws urging a separation between the anatomical features and applying a tension to the tissue.
